(19) United States
(12) Patent Application Publication
(10) Pub. No.: US 2025/0204917 A1
(43) Pub. Date: Jun. 26, 2025

(54) LEFT ATRIAL APPENDAGE CLOSURE DEVICE WITH ATRAUMATIC PECTINATE ANCHOR

(71) Applicant: Boston Scientific Scimed, Inc., Maple Grove, MN (US)

(72) Inventors: Joshua Mark Inouye, Brooklyn Park, MN (US); Scott Andrew McCullough, Plymouth, MN (US); Niraj Gurung, Monticello, MN (US); Graham Krumpelmann, Stillwater, MN (US)

(73) Assignee: Boston Scientific Scimed, Inc., Maple Grove, MN (US)

(21) Appl. No.: 18/987,019

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12

(52) U.S. Cl. CPC A61B 17/12122; A61B 17/12031; A61B 17/12159; A61B 2017/1205

(57) ABSTRACT

A left atrial appendage closure (LAAC) device includes an expandable occlusive element movable from a collapsed configuration for delivery to an expanded configuration for deployment. The expandable occlusive element may be an expandable foam element. An atraumatic anchor is engaged with the expandable occlusive element and is adapted to engage pectinate within the left atrial appendage (LAA) in order to secure the expandable occlusive element within the LAA.

### CROSS REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for closing off the left atrial appendage. An example may be found in a left atrial appendage closure (LAAC) device. The LAAC device includes an expandable occlusive element movable from a collapsed configuration for delivery to an expanded configuration for deployment and an atraumatic anchor engaged with the expandable occlusive element, the atraumatic anchor adapted to engage pectinate within the left atrial appendage (LAA) in order to secure the expandable occlusive element within the LAA.

Alternatively or additionally, the expandable occlusive element may include a foam element.

Alternatively or additionally, the collapsed configuration may correspond to the foam element being constrained within a delivery device and the expanded configuration may correspond to the foam element expanding to a biased configuration when no longer constrained by the delivery device.

Alternatively or additionally, the atraumatic anchor may include a proximal base portion that wraps around the expandable occlusive element in order to secure the atraumatic anchor to the expandable occlusive element.

Alternatively or additionally, the atraumatic anchor may include a proximal base portion that extends within the expandable occlusive element in order to secure the atraumatic anchor to the expandable occlusive element.

Alternatively or additionally, the atraumatic anchor may include a proximal base having an inner surface adapted to engage the expandable occlusive element and an outer surface that is threaded in order to threadedly engage a threaded sleeve forming part of a delivery device.

Alternatively or additionally, the atraumatic anchor may include a proximal base having an inner surface adapted to engage the expandable occlusive element and an outer surface including two or more slots adapted to engage with a torquer element forming part of a delivery device.

Alternatively or additionally, the atraumatic anchor may include a helical coil having an atraumatic tip.

Alternatively or additionally, the helical coil may include one or more retention bumps.

Alternatively or additionally, the helical coil may have a distally increasing diameter.

Alternatively or additionally, the atraumatic anchor may include two or more intertwined helical coils.

Alternatively or additionally, the two or more intertwined helical coils may each have a constant diameter.

Alternatively or additionally, the two or more intertwined helical coils may each have a distally increasing diameter.

Alternatively or additionally, the atraumatic anchor may include a plurality of curved members extending radially outwardly from a central point.

Alternatively or additionally, the plurality of curved members may define a proximally increasing diameter.

Alternatively or additionally, the LAAC device may further include an expandable coating on at least part of the atraumatic anchor.

Alternatively or additionally, the expandable occlusive element may be keyed to a delivery device component.

Another example may be found in a left atrial appendage closure (LAAC) device. The LAAC device includes an expandable foam element movable from a collapsed configuration for delivery to an expanded configuration for deployment and an atraumatic anchor engaged with the expandable foam element, the atraumatic anchor adapted to engage pectinate within the left atrial appendage (LAA) in order to secure the expandable occlusive element within the LAA.

Alternatively or additionally, the atraumatic anchor may include one or more helical coils.

Another example may be found in a left atrial appendage closure (LAAC) device. The LAAC device may include an expandable foam element movable from a collapsed configuration for delivery to an expanded configuration for deployment and an atraumatic anchor engaged with the expandable foam element. The atraumatic anchor includes a base portion adapted to be secured to the expandable foam element, a coil adapted to extend distally from the base portion and engage with pectinate within the left atrial appendage (LAA), and an atraumatic tip secured to a distal end of the coil.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cross-sectional view of an LAA (left atrial appendage);

FIG. 2A is a perspective view of an illustrative LAAC device in a compressed configuration while FIG. 2B is a perspective view of the illustrative LAAC device in an expanded configuration;

FIGS. 3A, 3B and 3C depict implantation of the illustrative LAAC (left atrial appendage closure device) of FIGS. 2A and 2B within the LAA;

FIG. 4A is a perspective view of an illustrative atraumatic anchor;

FIG. 4B is a perspective view of an illustrative atraumatic anchor;

FIGS. 5A through 5F are schematic views of illustrative atraumatic tips;

FIG. 6A is a perspective view of an illustrative LAAC device;

FIG. 6B is a perspective view of an illustrative LAAC device;

FIGS. 7A through 7D are perspective view of illustrative LAAC devices;

FIG. 8A is a schematic view of an illustrative LAAC device having a flexible coupling;

FIG. 8B is a schematic view of the illustrative LAAC device of FIG. 8A shown within the LAA;

FIG. 9A is a schematic view of an illustrative LAAC device having a variable pitch;

FIG. 9B is a schematic view of an illustrative LAAC device of FIG. 9A shown within the LAA;

FIG. 10A is a schematic view of an illustrative atraumatic anchor with an expandable material applied to the atraumatic anchor, with the expandable material shown in an initial, non-expanded configuration;

FIG. 10B is a schematic view of the illustrative atraumatic anchor of FIG. 10A, with the expandable material shown in a subsequent expanded configuration;

FIGS. 11A through 11G are schematic views showing an illustrative delivery method; and

FIGS. 12A through 12C are schematic views showing an illustrative delivery method.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

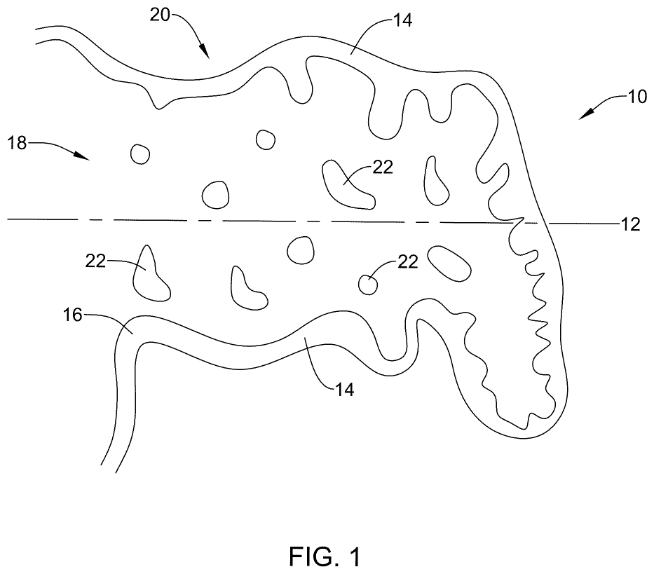

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

The LAA 10 shown in FIG. 1 is merely illustrative, as in some instances the LAA 10 may take a variety of different shapes. For example, the proximal mouth 18 may have a variety of different diameters and shapes, depending on the particular patient. In some instances, the LAA 10 may have a shallower depth than what is shown. In some instances, the LAA 10 may include lobes. In some instances, it is beneficial to provide left atrial appendage closure (LAAC) devices that are designed and adapted to accommodate a patient's LAA 10, regardless of the size and shape of the LAA 10. In some instances, the LAA 10 may include various structures within the LAA 10, such as but not limited to pectinate muscles 22. In some instances, the structures within the LAA 10, including the pectinate muscles 22, may provide a way to anchor devices within or near the LAA 10 without needing to puncture the lateral wall 14 of the LAA 10.

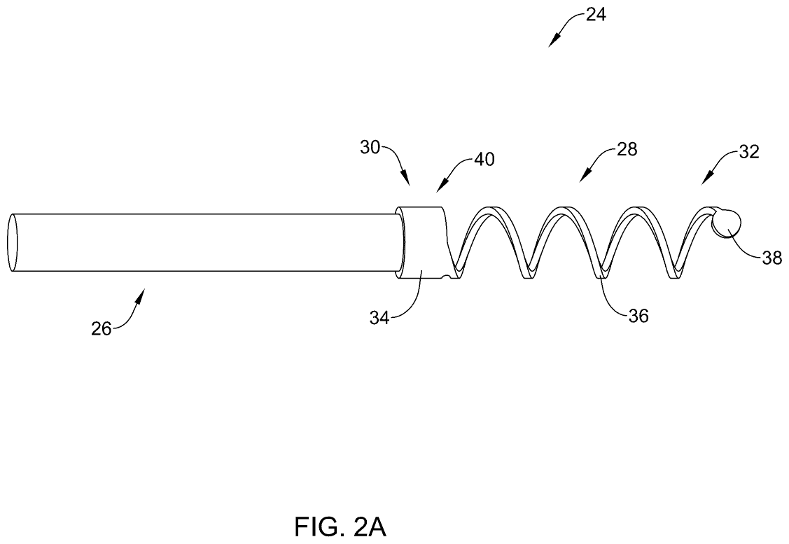

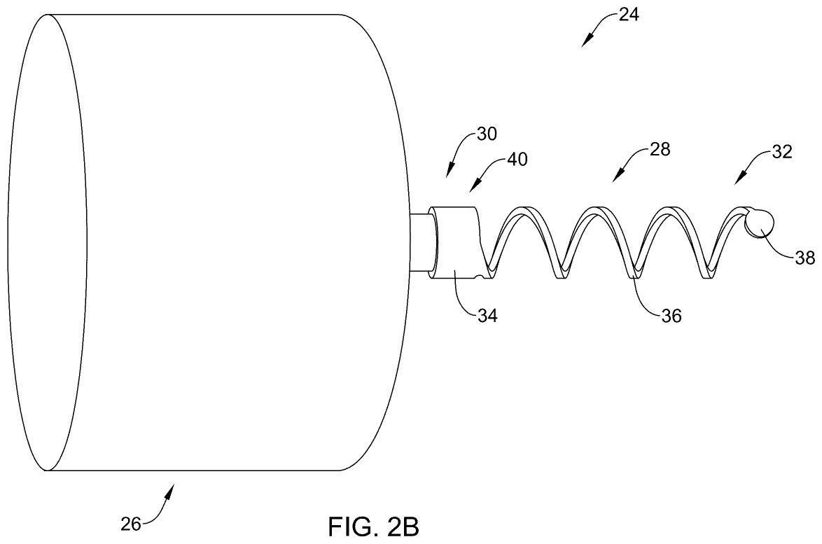

FIG. 2A is a perspective view of an illustrative LAAC device 24 that may be used in closing off the LAA 10. In some instances, the LAAC device 24 may itself close off the LAA 10. In some instances, the LAAC device 24 may partially close off the LAA 10, and subsequent tissue growth may help to more completely close off the LAA 10. By closing off the LAA 10, the risk of blood clots forming and breaking loose from within the LAA 10 may be dramatically reduced. The LAAC device 24 includes an expandable occlusive element 26 that is movable from a collapsed configuration (as shown in FIG. 2A) for delivery to an expanded configuration (as shown in FIG. 2B) for deployment. The expandable occlusive element 26 may be formed of any of a variety of different materials, as long as the materials are capable of moving from the collapsed configuration to the expanded configuration. As an example, the collapsed configuration may correspond to the foam element being constrained within a delivery device and the expanded configuration may correspond to the foam element expanding to a biased configuration when no longer constrained by the delivery device.

In some instances, the expandable occlusive element 26 may have a “remembered” configuration that corresponds to the expanded configuration, and is adapted to be able to be compressed or otherwise constrained in the collapsed configuration, and then subsequently regain the expanded configuration once the expandable occlusive element 26 is no longer constrained in the collapsed configuration. In some instances, the expandable occlusive element 26 may be made of a foam. A foam may be made from a variety of different polymeric materials, and includes a large number of void spaces within the foam, particularly in the expanded configuration. In some instances, these void spaces allow the foam to be easily compressed down into the compressed configuration.

The LAAC device 24 includes an atraumatic anchor 28 that is engaged with the expandable occlusive element 26. In some instances, the atraumatic anchor 28 may be adapted to be able to engage the pectinate muscle 22 within the LAA 10 in order to anchor the LAAC device 24 in position relative to the LAA 10. In some instances, the atraumatic anchor 28 may be adapted to be able to anchor the LAAC device 24 in position relative to the LAA 10 without puncturing or substantially penetrating into the lateral wall 14 of the LAA 10. The atraumatic anchor 28 may provide a less traumatic securement for the LAAC device 24. In some instances, the atraumatic anchor 28 may be considered as extending from a proximal region 30 to a distal region 32. The atraumatic anchor 28 may include a base portion 34 that couples the atraumatic anchor 28 to the expandable occlusive element 26, a coil portion 36 that has a helical or coiled profile, and an atraumatic tip 38. In some instances, as shown, the atraumatic anchor 28 may be laser-cut from a cylinder of appropriate material. In some instances, the atraumatic anchor 28 may be formed from a coiled wire, for example.

The atraumatic anchor 28 may be formed of any suitable material. In some instances, the atraumatic anchor 28 may be formed of a metal such as nitinol or stainless steel. In some instances, the atraumatic anchor 28 may be formed of an injection molded polymer. The atraumatic anchor 28 may be formed via a polymer extrusion process, for example. In some instances, the atraumatic anchor 28 may have a composite construction. As an example, the atraumatic anchor 28 may have a metallic core covered by a polymer over-mold or coating. As another example, the atraumatic anchor 28 may be formed as separate parts and then combined together, such as the base portion 34, the coil portion 36 and the atraumatic tip 38 separately formed and then combined together to form the atraumatic anchor 28. As an example, the base portion 34, the coil portion 36 and the atraumatic tip 38 may be soldered or welded together. Adhesives may be used in joining together one or more of the base portion 34, the coil portion 36 and the atraumatic tip 38.

The base portion 34 allows the atraumatic anchor 28 to be secured to the expandable occlusive element 26. In some instances, the base portion 34 may form a frictional or compressive fit with a distal portion 40 of the expandable occlusive element 26. In some instances, the base portion 34 may be adhesively secured to the expandable occlusive element 26, with the distal portion 40 of the expandable occlusive element 26 disposed within the base portion 34 of the atraumatic anchor 28. In some instances, particularly if the atraumatic anchor 28 is formed from a coiled wire, the base portion 34 may wrap tightly around the distal portion 40 of the expandable occlusive element 26 in order to secure the atraumatic anchor 28 to the expandable occlusive element 26. In some instances, while not shown, the atraumatic anchor 28 may extend into an interior of the expandable occlusive element 26 and thus may form a mechanical lock between the atraumatic anchor 28 and the expandable occlusive element 26.

The coil portion 36 extends distally from the base portion 34 and is adapted to engage the pectinate muscle 22 within the LAA 10. In some instances, the atraumatic anchor 28 is rotated or twisted upon implantation in order to allow the coil portion 36 to engage the pectinate muscle 22 within the LAA 10 and thus pull the atraumatic anchor 28 further into the LAA 10 as the coil portion 36 interacts with the pectinate muscle 22 in a threaded manner. In some instances, the coil portion 36 may have a length that is sufficient to allow the atraumatic anchor 28 to extend into the LAA 10 without puncturing the lateral wall 14 of the LAA 10. As an example, the atraumatic anchor 28 as a whole may have an overall length in a range of about 3 millimeters to about 20 millimeters, with the coil portion 36 consuming seventy to ninety percent of the overall length of the atraumatic anchor 28. In some instances, the coil portion 36 may be considered as having a diameter and a pitch that allows the coil portion 36 to become entangled in the pectinate muscle 22. As an example, the coil portion 36 may have a diameter in a range of about 2 millimeters to about 40 millimeters, and may have a pitch (defined as an axial distance between adjacent windings) that is in a range of about 1 millimeter to about 20 millimeters. Other relative dimensions are also contemplated.

The atraumatic tip 38 is attached to the coil portion 36 within the distal region 32 of the atraumatic anchor 28. In some instances, the atraumatic tip 38 forms the distalmost portion of the atraumatic anchor 28. The atraumatic tip 38 may take a variety of forms, as will be shown in subsequent drawings, and may be adapted to guide the atraumatic anchor 28 into the LAA 10, through the pectinate muscles 22, without damaging the pectinate muscles 22 and/or any other portion of the LAA 10. In some instances, the atraumatic tip 38 may be adapted to, when hitting up against tissue such as the pectinate muscles 22, push the tissue to the side of the atraumatic tip 38 without damaging the tissue. As a result, the atraumatic anchor 28 is able to continue to rotate relative to the pectinate muscles 22, and thus the atraumatic anchor 28 is able to pull the LAAC device 24 further into the LAA 10.

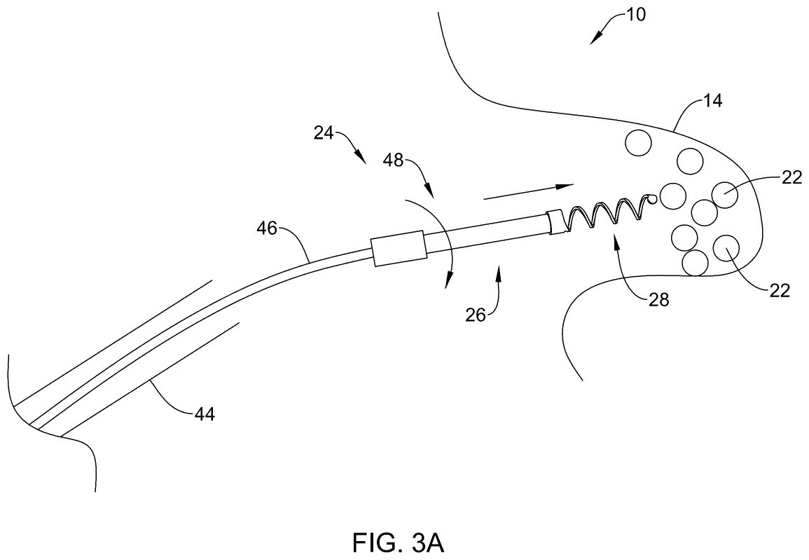

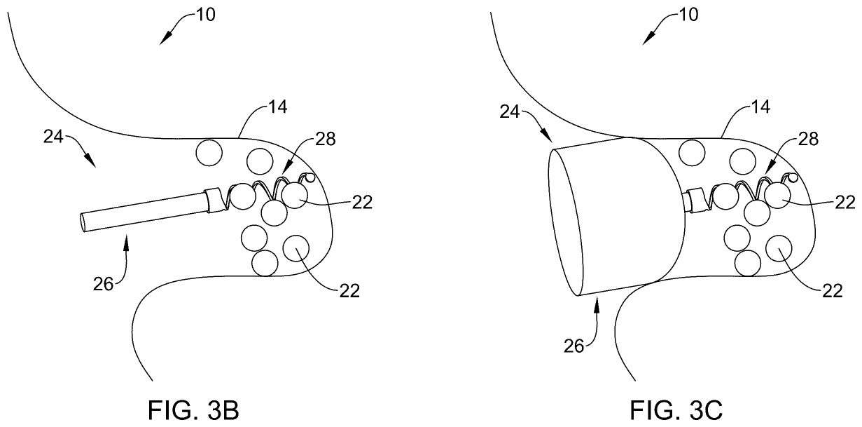

FIGS. 3A, 3B and 3C provide a schematic example of implanting the LAAC device 24. In FIG. 3A, the LAAC device 24 can be seen loaded within a delivery device 42. The delivery device 42 includes an outer sheath 44 and a torquer element 46 that has been advanced distally relative to the outer sheath 44 (or the outer sheath 44 has been withdrawn proximally relative to the torquer element 46. The torquer element 46 is engaged with a proximal region 48 of the expandable occlusive element 26. While not shown, in some instances the torquer element 46 may be keyed to the expandable occlusive element 26 such that rotation of the torquer element 46 causes rotation of the expandable occlusive element 26, which in turn leads to rotation of the atraumatic anchor 28. In FIG. 3B, the delivery device 42 has been disengaged from the LAAC device 24, leaving the LAAC device 24 within the LAA 10 with the atraumatic anchor 28 engaged within the pectinate muscle 22. Over time, the expandable occlusive element 26 expands from its collapsed configuration as shown in FIG. 3B to its expanded configuration as shown in FIG. 3C. In some instances, the expandable occlusive element 26 may expand quickly once the outer sheath 44 has been withdrawn and the torquer element 46 has been disengaged from the expandable occlusive element 26. In some instances, exposure to fluid within the LAA 10 may facilitate the expandable occlusive element 26 expanding.

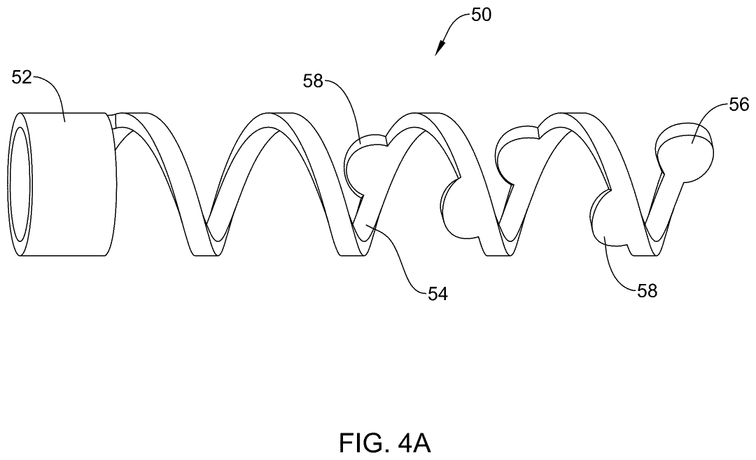

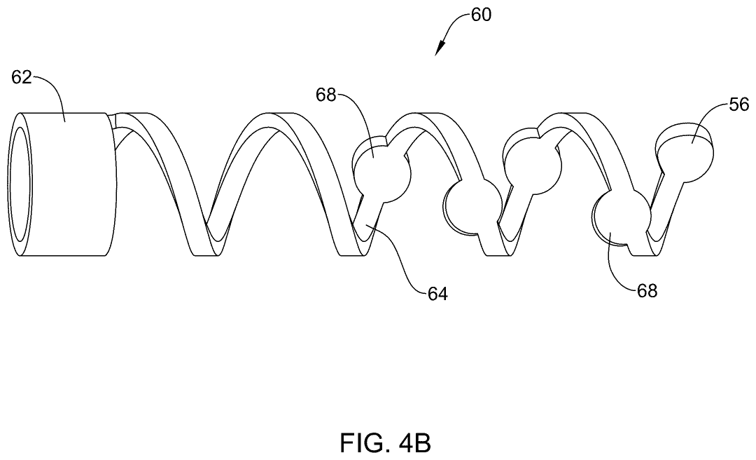

As noted, the atraumatic anchor 28 may take a variety of forms. FIG. 4A is a perspective view of an illustrative atraumatic anchor 50. The atraumatic anchor 50 may be considered as an alternative to the atraumatic anchor 28. The atraumatic anchor 50 includes a base portion 52, a coil portion 54 and an atraumatic tip 56. In this example, the atraumatic anchor 50 has been laser cut from a cylinder of a suitable material. As shown, the coil portion 54 includes a number of retention bumps 58 that extend circumferentially from the coil portion 54 and are curved to match the original shape of the cylinder from which the atraumatic anchor 50 was cut. In this example, the retention bumps 58 may be considered as being one sided, with one side of each retention bump 58 matching the profile of the coil portion 54 and an opposing side of each retention bump 58 curving out away from the profile of the coil portion 54. The retention bumps 58 help the atraumatic anchor 56 to gather purchase around the pectinate muscles 22. The atraumatic tip 56 also has a curved profile matching that of the cylinder from which the atraumatic anchor 50 was cut.

FIG. 4B is a perspective view of an illustrative atraumatic anchor 60. The atraumatic anchor 60 may be considered as an alternative to the atraumatic anchor 28. The atraumatic anchor 60 includes a base portion 62, a coil portion 64 and an atraumatic tip 66. In this example, the atraumatic anchor 60 has been laser cut from a cylinder of a suitable material. As shown, the coil portion 64 includes a number of retention bumps 68 that extend circumferentially from the coil portion 64 and are curved to match the original shape of the cylinder from which the atraumatic anchor 60 was cut. In this example, the retention bumps 68 may be considered as being two sided, with each side of each retention bump 68 curving out away from the profile of the coil portion 64. The retention bumps 68 help the atraumatic anchor 66 to gather purchase around the pectinate muscles 22. The atraumatic tip 66 also has a curved profile matching that of the cylinder from which the atraumatic anchor 60 was cut.

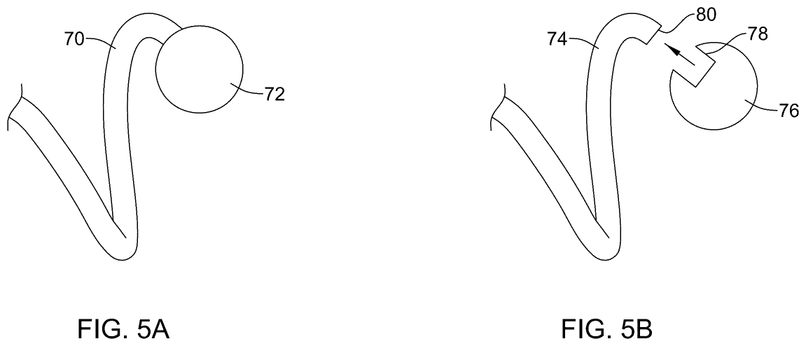

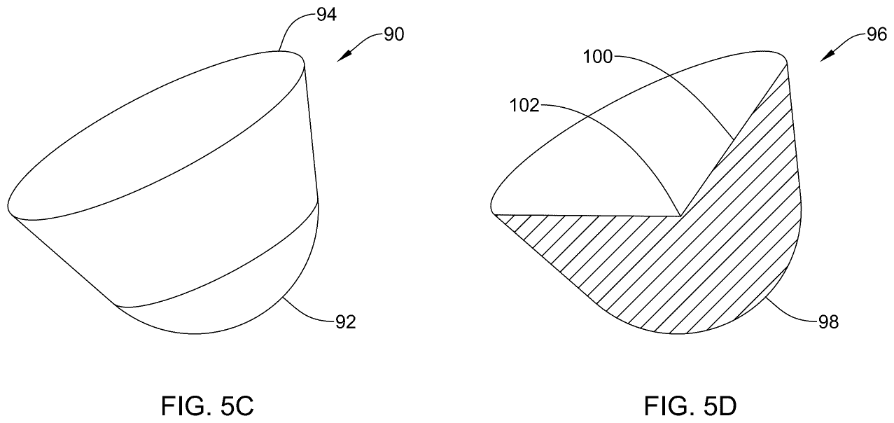

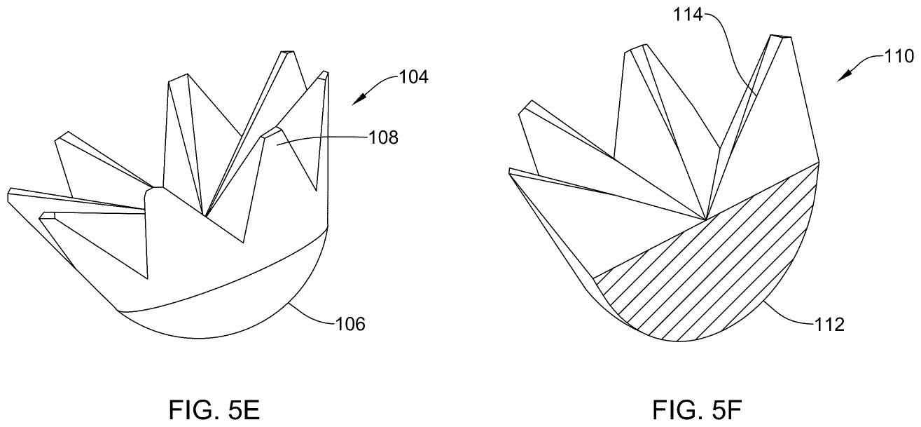

As noted, the atraumatic tip may take a variety of forms. FIGS. 5A through 5F provide several examples. In FIG. 5A, a portion of an atraumatic anchor 70 includes a radiopaque ball 72 as the atraumatic tip. It will be appreciated that this will enhance visibility of the atraumatic tip during fluoroscopic imaging. The radiopaque ball 72 may be formed of, or coated with, any of a variety of different radiopaque materials such as barium sulfate, bismuth compounds, gold and tungsten. In some instances, various polymers may be made to be radiopaque upon the addition of particular materials to the polymer. The radiopaque ball 72 may be attached to the atraumatic anchor 70 using any of a variety of different techniques, including adhesives and welding. In FIG. 5B, a portion of an atraumatic anchor 74 includes a ball 76 that forms an atraumatic tip. The ball 76 may include a slot 78 that accommodates a distal end 80 of the atraumatic anchor 74. The ball 76 may be an example of the radiopaque ball 72 shown in FIG. 5A, for example. In some instances, the ball 76 may be secured to the atraumatic anchor 74 using an adhesive, a screw, or via over-molding. In some instances, the ball 76 may simply be a laser spot weld created at the distal end 80 of the atraumatic anchor 74.

In some instances, the tip geometry may not be spherical as shown in FIGS. 5A and 5B. As an example, a laser spot weld created at the distal end 80 of the atraumatic anchor 74 may instead be elliptical in shape. In some instances, the atraumatic tip may include backward facing structures that allow the distal edge of the atraumatic tip to remain atraumatic while enhancing the ability of the atraumatic anchor to engage the pectinate muscles 22. FIG. 5C shows an atraumatic tip 90 that includes a distally facing atraumatic surface 92 and a proximally facing lip 94 that may help to engage the pectinate muscles 22. FIG. 5D shows an atraumatic tip 96 that includes a distally facing atraumatic surface 98 and a proximally facing lip 100 that includes one or more distinct angular changes 102. FIG. 5E shows an atraumatic tip 104 that includes a distally facing atraumatic surface 106 and a number of proximally facing barbs 108 that may help to engage the pectinate muscles 22. While FIG. 5E shows the atraumatic tip 104 as extending 360 degrees around in symmetric fashion, FIG. 5F shows an asymmetric atraumatic tip 110 that includes a distally facing atraumatic surface 112 and a number of proximally facing barbs 114 that do not extend 360 degrees around in symmetric fashion.

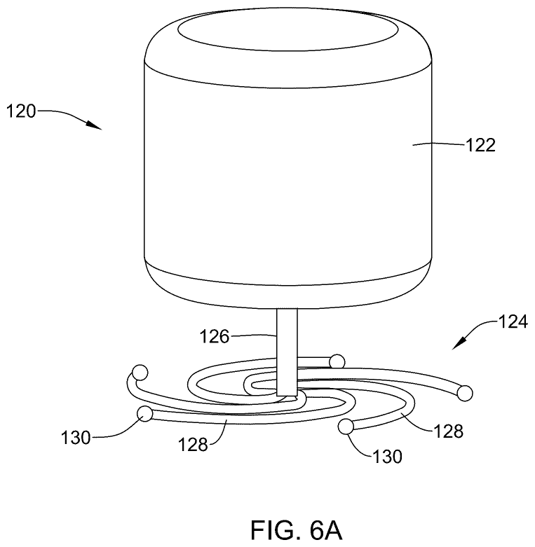

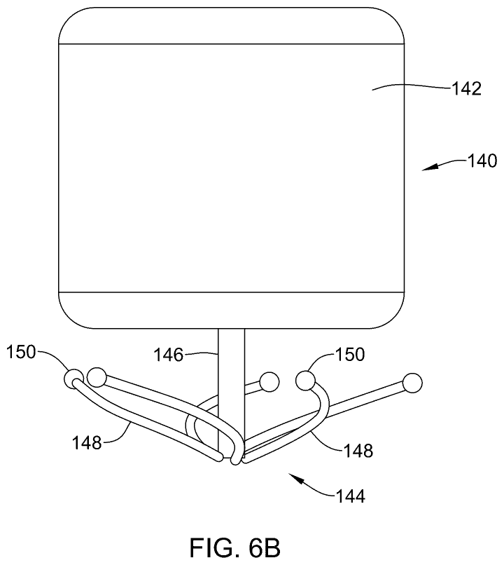

FIG. 6A is a perspective view of an illustrative LAAC device 120 that includes an expandable occlusive element 122 and an atraumatic anchor 124. The atraumatic anchor 124 includes a central shaft 126. In some instances, the central shaft 126 may extend into an interior of the expandable occlusive element 122. The atraumatic anchor 124 includes a number of curved members 128 that extend radially outwardly from the central shaft 126. While a total of five curved members 128 are shown, this is merely illustrative, as the atraumatic anchor 124 may include any number of curved members 128. In some instances, increasing the number of curved members 128 may provide more opportunities for the atraumatic anchor 124 to catch pectinate muscles 22. In some instances, as shown, each of the curved members 128 may extend within a single plane. This may facilitate use of the LAAC device 120 within a shallower LAA 10. The curved members 128 extending within a single plane may facilitate engaging pectinate muscles 22 that are axially oriented. Each of the curved members 128 may include an atraumatic tip 130. While the atraumatic tips 130 are shown as being spherical, this is merely illustrative, as the atraumatic tips 130 may take the form of any of the atraumatic tips described herein.

FIG. 6B is a perspective view of an illustrative LAAC device 140 that includes an expandable occlusive element 142 and an atraumatic anchor 144. The atraumatic anchor 144 includes a central shaft 146. In some instances, the central shaft 146 may extend into an interior of the expandable occlusive element 142. The atraumatic anchor 144 includes a number of curved members 148 that extend radially outwardly from the central shaft 146. While a total of five curved members 148 are shown, this is merely illustrative, as the atraumatic anchor 144 may include any number of curved members 148. In some instances, as shown, each of the curved members 148 may extend at least partially in a proximal direction, towards the expandable occlusive element 142. In some instances, having the curved members 148 curve proximally can provide a spring loading that holds or pushes the LAAC device 140 distally. Each of the curved members 148 may include an atraumatic tip 140. While the atraumatic tips 140 are shown as being spherical, this is merely illustrative, as the atraumatic tips 140 may take the form of any of the atraumatic tips described herein.

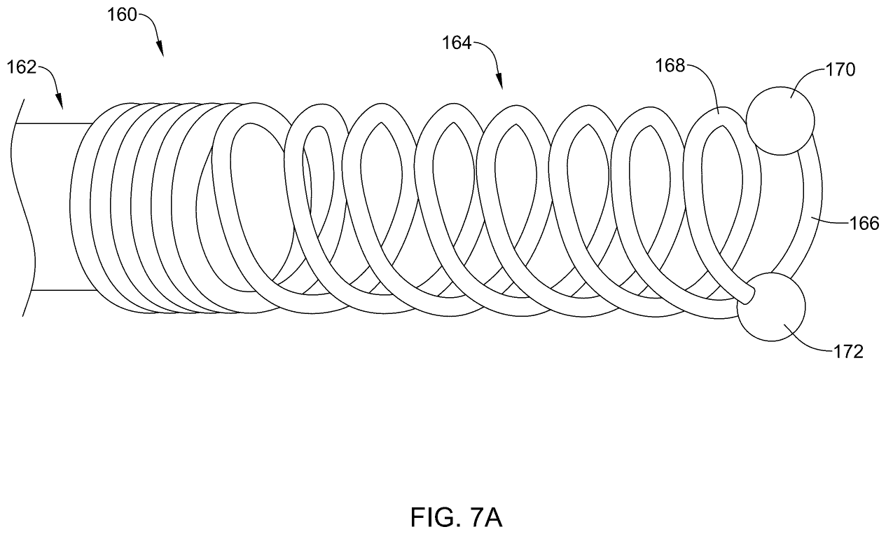

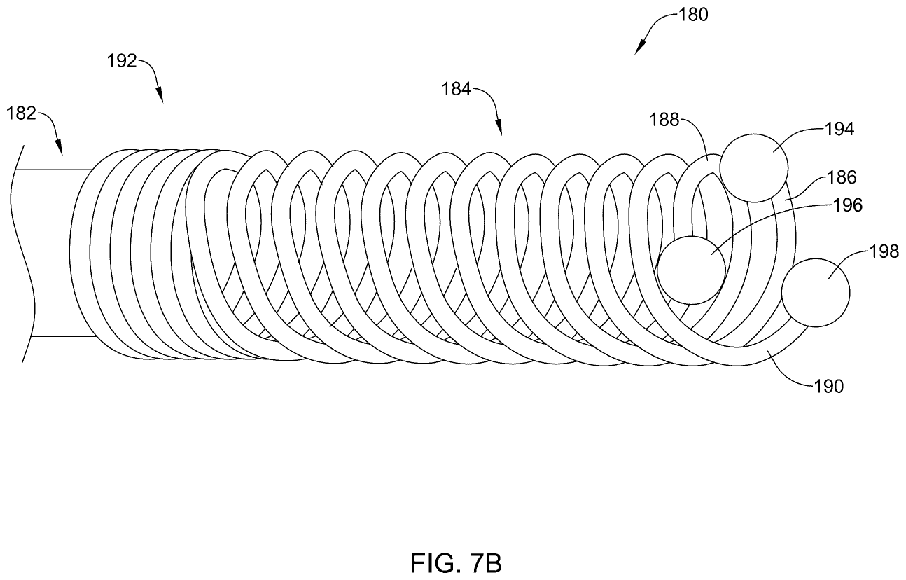

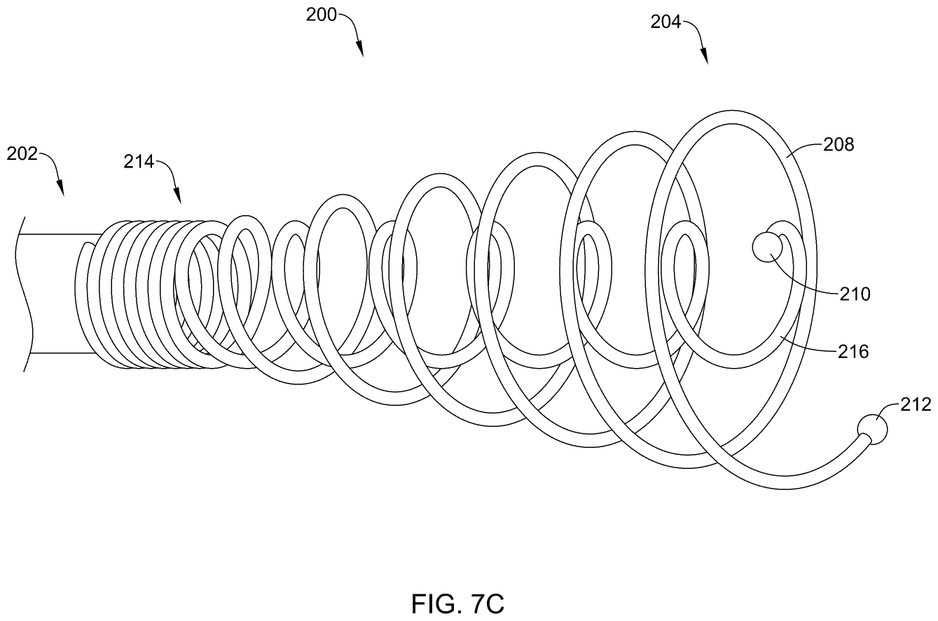

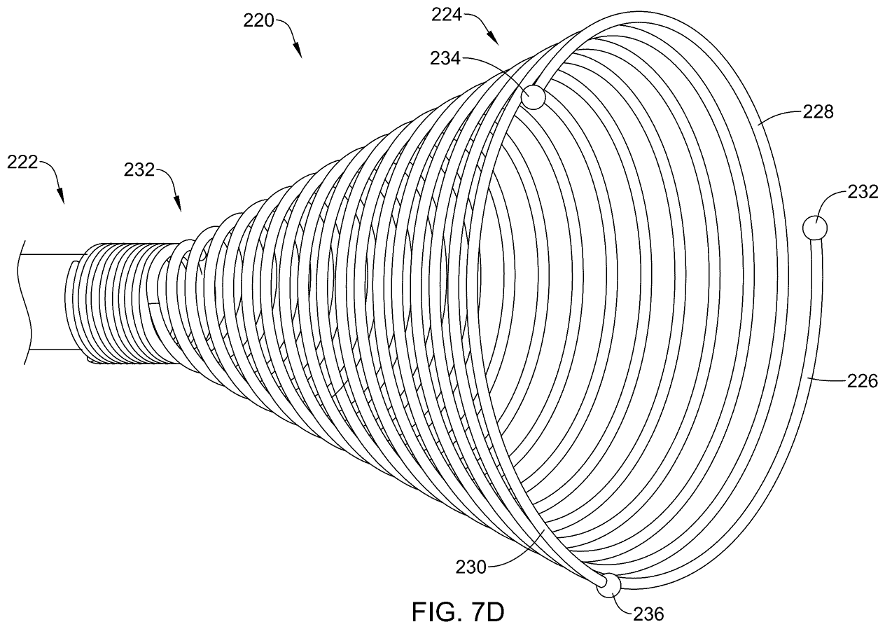

FIG. 7A is a schematic view of an illustrative LAAC device 160 that includes an expandable occlusive element 162 and an atraumatic anchor 164. The atraumatic anchor 164 includes a first helix 166 and a second helix 168. Having two or more intertwined helices can provide better grabbing of the pectinate muscles 22. Each of the first helix 166 and the second helix 168 have a constant diameter and a constant pitch, apart from a proximal portion 170 of the first helix 166 and the second helix 168 where the first helix 166 and the second helix 168 are wrapped tightly around the expandable occlusive element 162 in order to secure the expandable occlusive element 162 to the atraumatic anchor 164. The first helix 166 includes an atraumatic tip 170 and the second helix 168 includes an atraumatic tip 172. While the atraumatic tips 170 and 172 are shown as being spherical, this is merely illustrative, as the atraumatic tips 170 and 172 may take the form of any of the atraumatic tips described herein.

FIG. 7B is a schematic view of an illustrative LAAC device 180 that includes an expandable occlusive element 182 and an atraumatic anchor 184. The atraumatic anchor 184 includes a first helix 186, a second helix 188 and a third helix 190. Having two or more intertwined helices can provide better grabbing of the pectinate muscles 22. Each of the first helix 186, the second helix 188 and the third helix 190 have a constant diameter and a constant pitch, apart from a proximal portion 192 of the first helix 186, the second helix 188 and the third helix 190 where the first helix 186, the second helix 188 and the third helix 190 are wrapped tightly around the expandable occlusive element 182 in order to secure the expandable occlusive element 182 to the atraumatic anchor 184. The first helix 186 includes an atraumatic tip 194, the second helix 188 includes an atraumatic tip 196 and the third helix 190 includes an atraumatic tip 198. While the atraumatic tips 194, 196 and 198 are shown as being spherical, this is merely illustrative, as the atraumatic tips 194, 196 and 198 may take the form of any of the atraumatic tips described herein.

FIG. 7C is a schematic view of an illustrative LAAC device 200 that includes an expandable occlusive element 202 and an atraumatic anchor 204. The atraumatic anchor 204 includes a first helix 206 that has a constant diameter and a constant pitch, and a second helix 208 that has a distally increasing diameter and a constant pitch, apart from a proximal portion 214 of the first helix 206 and the second helix 208 wherein the first helix 206 and the second helix 208 are wrapped tightly around the expandable occlusive element 202 in order to secure the expandable occlusive element 202 to the atraumatic anchor 204. In some instances, one or more of the first helix 206 and the second helix 208 may include at least a portion thereof that instead has a proximally increasing diameter. As an example, a proximal half of one or more of the first helix 206 and the second helix 208 may have a distally increasing diameter while a distal half of one or more of the first helix 206 and the second helix 208 may have a proximally increasing diameter. Having two or more intertwined helices can provide better grabbing of the pectinate muscles 22. As shown, the first helix 206 extends within the second helix 208. The first helix 206 includes an atraumatic tip 210 and the second helix 208 includes an atraumatic tip 212. While the atraumatic tips 210 and 212 are shown as being spherical, this is merely illustrative, as the atraumatic tips 210 and 212 may take the form of any of the atraumatic tips described herein.

FIG. 7D is a schematic view of an illustrative LAAC device 220 that includes an expandable occlusive element 222 and an atraumatic anchor 224. The atraumatic anchor 224 includes a first helix 226, a second helix 228 and a third helix 230. Having two or more intertwined helices can provide better grabbing of the pectinate muscles 22. Each of the first helix 226, the second helix 228 and the third helix 230 have a distally increasing diameter and a constant pitch, apart from a proximal portion 232 of the first helix 226, the second helix 228 and the third helix 230 where the first helix 226, the second helix 228 and the third helix 230 are wrapped tightly around the expandable occlusive element 222 in order to secure the expandable occlusive element 222 to the atraumatic anchor 224. The first helix 226 includes an atraumatic tip 232, the second helix 228 includes an atraumatic tip 234 and the third helix 230 includes an atraumatic tip 236. While the atraumatic tips 232, 234 and 236 are shown as being spherical, this is merely illustrative, as the atraumatic tips 232, 234 and 236 may take the form of any of the atraumatic tips described herein.

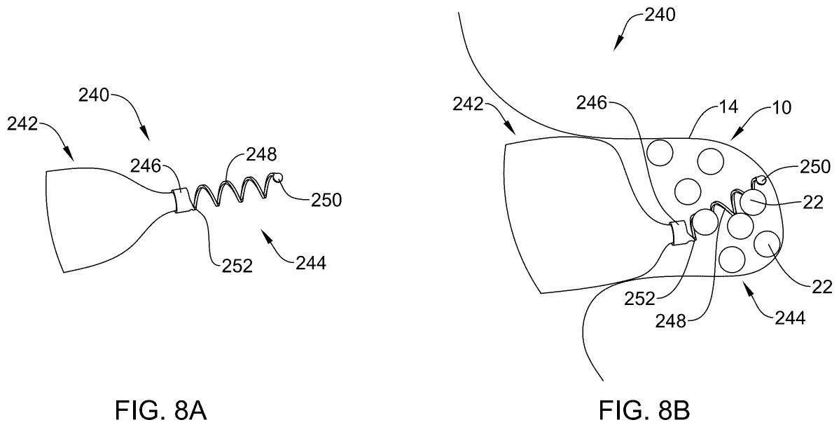

FIG. 8A is a schematic view of an illustrative LAAC device 240 that includes an expandable occlusive element 242 and an atraumatic anchor 244. The atraumatic anchor 244 includes a base portion 246 that secures the atraumatic anchor 244 to the expandable occlusive element 242. The atraumatic anchor 244 includes a coil portion 248 and an atraumatic tip 250. In some instances, as shown, the atraumatic anchor 244 includes a flexible portion 252 disposed between the base portion 246 and the coil portion 248. In some instances, as shown for example in FIG. 8B, which shows the LAAC device 240 disposed within the LAA 10, the flexible portion 252 allows the atraumatic anchor 244 to flex to the side as it gets tangled in the pectinate muscles 22. In some instances, this provides additional flexibility in the approach angle that the LAAC device 240 needs to approach the LAA 10 in order to capture the pectinate muscle 22 while allowing the expandable occlusive element 242 to sit appropriately within the LAA 10.

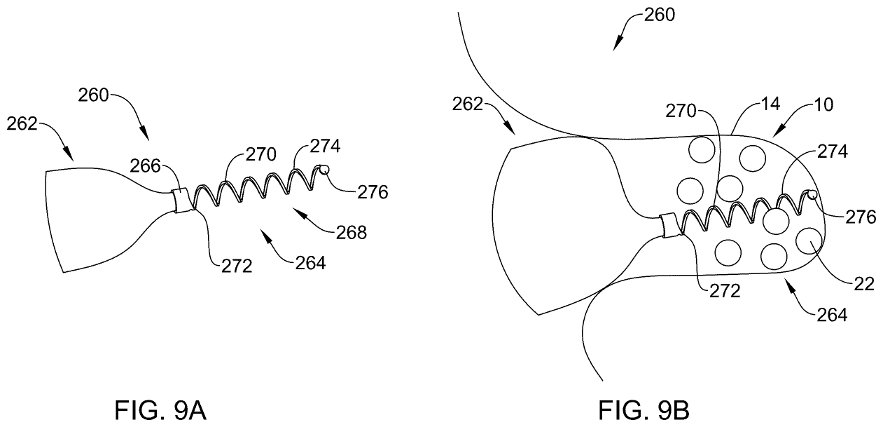

FIG. 9A is a schematic view of an illustrative LAAC device 260 that includes an expandable occlusive element 262 and an atraumatic anchor 264. The atraumatic anchor 264 includes a base portion 266 that secures the atraumatic anchor 264 to the expandable occlusive element 262. The atraumatic anchor 264 includes a coil portion 268 that includes a segment 270 with a tight pitch and several segments 274 and 276 that have a loose pitch. In some instances, the segments 274 and 276 with the loose pitch may facilitate engaging the pectinate muscle 22 while the segment 270 having the tight pitch may facilitate pinching the pectinate muscle 22 and thus helping to secure the LAAC device 260 in position. This is illustrated in FIG. 9B, which shows the LAAC device 260 disposed within the LAA 10.

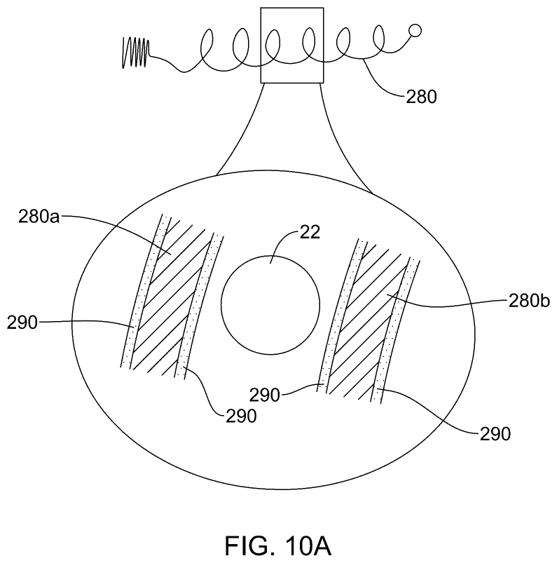

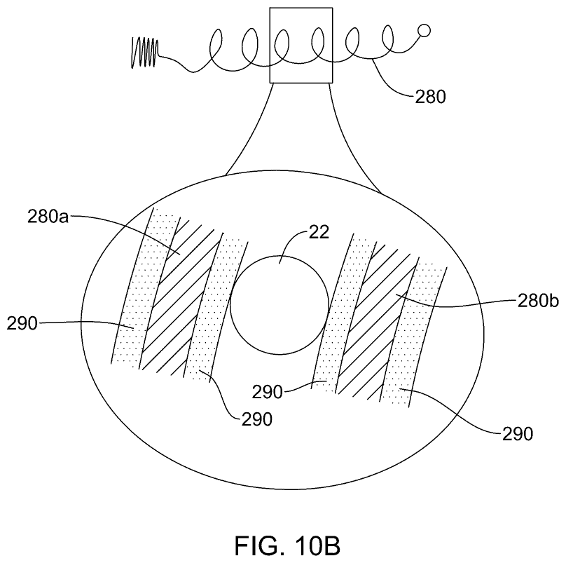

In some instances, a coating may be applied to the atraumatic anchor. FIG. 10A is a schematic view of a portion of a coil 280 having a first coil winding 280a on a first side of a pectinate muscle 22 and a second coil winding 280b on a second side of the pectinate muscle 22. In FIG. 10A, a coating 290 has been applied to each of the first coil winding 280a and the second coil winding 280b, but has not yet expanded or swelled. In some instances, the coating 290 may be a swellable polymeric coating. In some instances, the coating 290 may be a shape memory foam that has an initial low profile. In FIG. 10B, the coating 290 has swelled or returned to a larger profile. The coating 290 fills much of the space between the first coil winding 280a, the second coil winding 280b and the pectinate muscle 22. This helps to lock the coil 280 in place. In some instances, other coatings such as a lubricious hydrophilic coating may be applied to the atraumatic anchor to facilitate delivery.

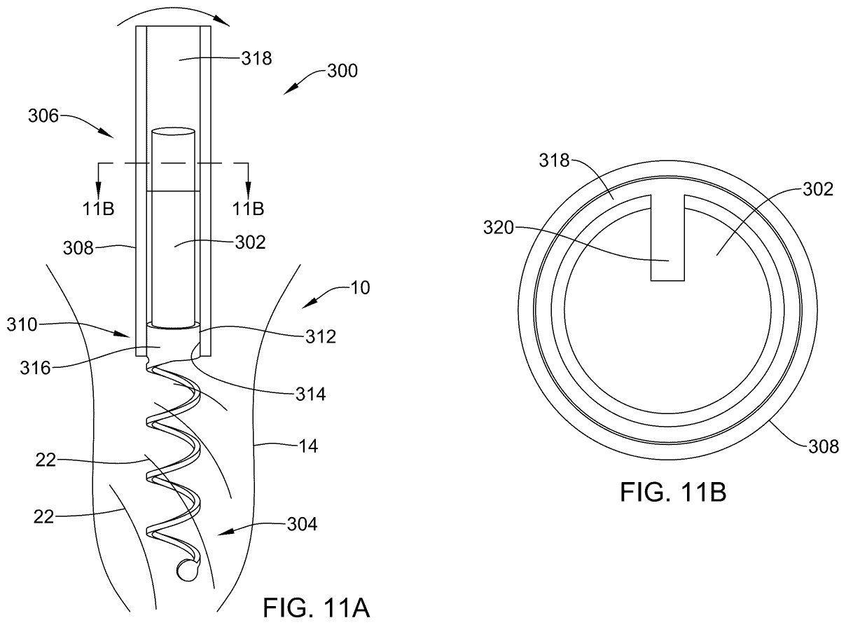

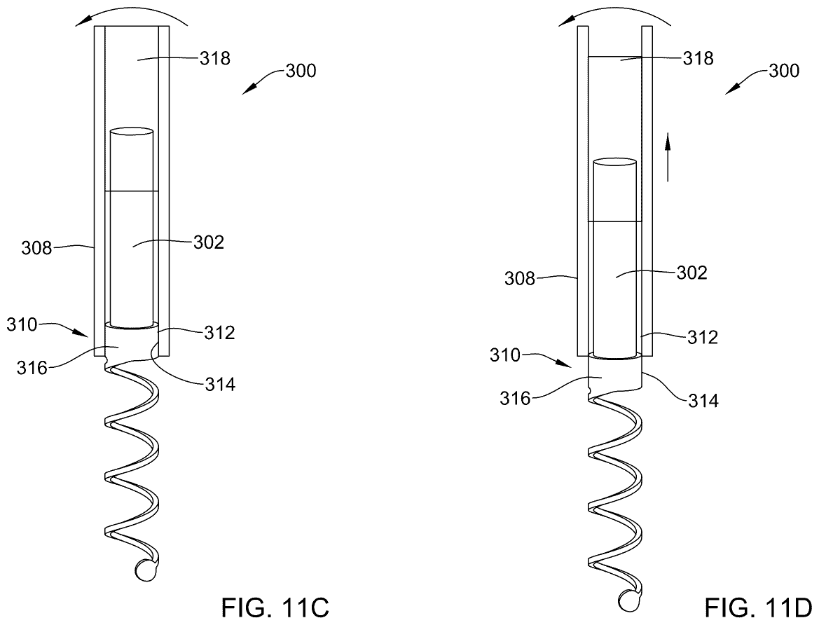

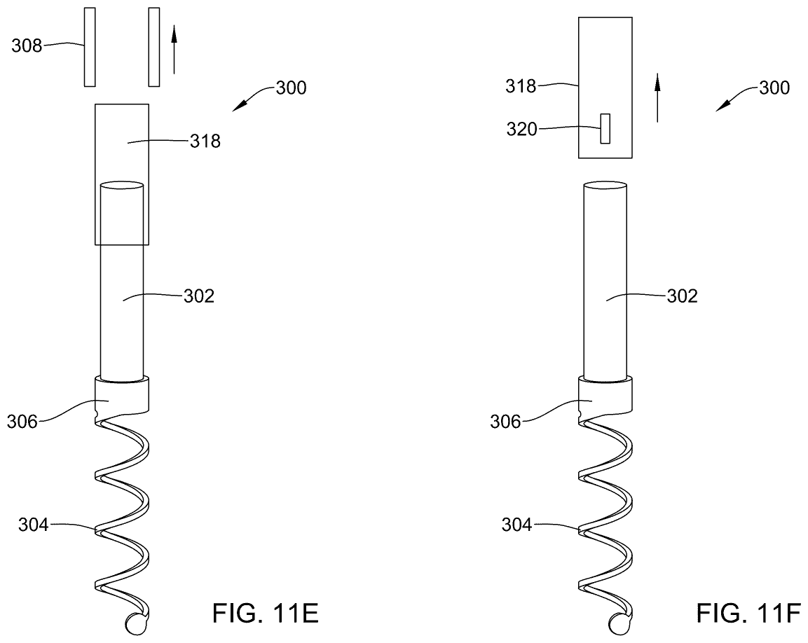

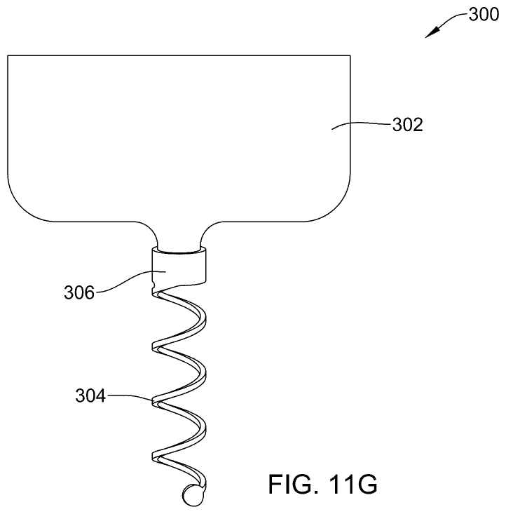

FIGS. 11A through 11G are schematic views showing an illustrative delivery method for delivering an LAAC device 300. In FIG. 11A, the LAAC device 300 includes an expandable occlusive element 302 and an atraumatic anchor 304 and is loaded into a delivery device 306. The delivery device 306, which is shown schematically, includes an outer threaded sleeve 308. A distal region 310 of the outer threaded sleeve 308 includes a threaded surface 312 that is adapted to threadedly engage an outer threaded surface 314 formed on a base portion 316 of the atraumatic anchor 304. A torquer component 318 engages the expandable occlusive element 302 and as shown in FIG. 11B, which is a cross-sectional view taken along line 11B-11B of FIG. 11A, the torquer component 318 includes a key 320 that engages the expandable occlusive element 302 such that rotation of the torquer component 318 results in rotation of the expandable occlusive element 302 and thus rotation of the atraumatic anchor 304.

Once the LAAC device 300 is engaged with the pectinate muscle 22 within the LAA 10, the delivery device 306 may be removed. As shown in FIGS. 11C and 11D, rotation of the outer threaded sleeve 308 relative to the atraumatic anchor 304 causes the outer threaded sleeve 308 to move proximally and away from the base portion 316 of the atraumatic anchor 304. Moving to FIG. 11E, it can be seen that the outer threaded sleeve 308 has been disengaged from the LAAC device 300 and can be withdrawn. In FIG. 11F, the torquer component 318 has been disengaged from the expandable occlusive element 302. Finally, in FIG. 11G, the expandable occlusive element 302 has reached its expanded configuration.

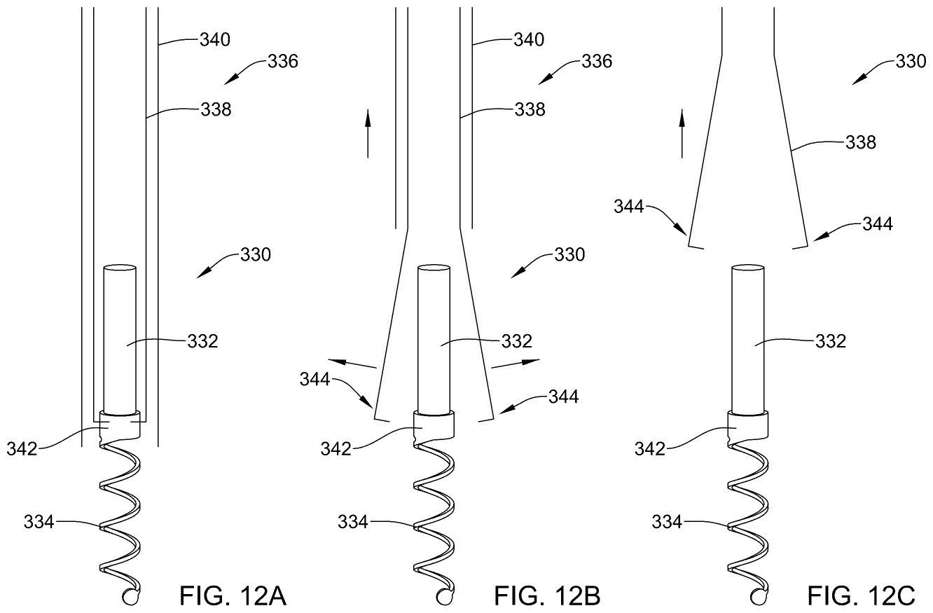

FIGS. 12A through 12C are schematic views showing an illustrative delivery method for delivering an LAAC device 330. In FIG. 12A, the LAAC device 330 includes an expandable occlusive element 332 and an atraumatic anchor 334 and is loaded into a delivery device 336. The delivery device 336 includes a torquer component 338 and an outer sleeve 340 that holds the torquer component 338 engaged with the atraumatic anchor 334 when the outer sleeve 340 is positioned as shown in FIG. 12A. In some instances, the atraumatic anchor 334 includes a base portion 342 that secures the expandable occlusive element 332 to the atraumatic anchor 334. The base portion 342 may include one or more slots (not visible) that allow a distal hook feature 344 of the torquer component 338 to engage the base portion 342. The distal hook feature 344 may include two halves, two shorter strips or four shorter strips, for example. Once the LAAC device 330 has reached a desired position relative to the LAA 10, the delivery device 336 can be removed. As shown in FIG. 12B, the outer sleeve 340 has been withdrawn proximally, which allows the distal hook feature 344 of the torquer component 338 to release the base portion 342 of the atraumatic anchor 334. Moving to FIG. 12C, the outer sleeve 340 has been completely withdrawn, and the torquer component 338 has released the LAAC device 330. While not shown, the expandable occlusive element 332 may now expand to its expanded configuration.

The materials that can be used for the various components of the LAAC devices described herein, and the various elements thereof, disclosed herein may include those commonly associated with medical devices. In some instances, the devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the LAAC devices, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the LAAC devices and/or other elements disclosed herein. For example, the devices, and/or components or portions thereof, may be made of a material that does not substantially distort the image and create substantial artifacts (e.g., gaps in the image). Certain ferromagnetic materials, for example, may not be suitable because they may create artifacts in an MRI image. The LAAC devices, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the LAAC devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A left atrial appendage closure (LAAC) device, comprising:
an expandable occlusive element movable from a collapsed configuration for delivery to an expanded configuration for deployment; and
an atraumatic anchor engaged with the expandable occlusive element, the atraumatic anchor adapted to engage pectinate within the left atrial appendage (LAA) in order to secure the expandable occlusive element within the LAA.

2. The LAAC device of claim 1, wherein the expandable occlusive element comprises a foam element.

3. The LAAC device of claim 2, wherein the collapsed configuration corresponds to the foam element being constrained within a delivery device and the expanded configuration corresponds to the foam element expanding to a biased configuration when no longer constrained by the delivery device.

4. The LAAC device of claim 1, wherein the atraumatic anchor comprises a proximal base portion that wraps around the expandable occlusive element in order to secure the atraumatic anchor to the expandable occlusive element.

5. The LAAC device of claim 1, wherein the atraumatic anchor comprises a proximal base portion that extends within the expandable occlusive element in order to secure the atraumatic anchor to the expandable occlusive element.

6. The LAAC device of claim 1, wherein the atraumatic anchor comprises a proximal base having an inner surface adapted to engage the expandable occlusive element and an outer surface that is threaded in order to threadedly engage a threaded sleeve forming part of a delivery device.

7. The LAAC device of claim 1, wherein the atraumatic anchor comprises a proximal base having an inner surface adapted to engage the expandable occlusive element and an outer surface including two or more slots adapted to engage with a torquer element forming part of a delivery device.

8. The LAAC device of claim 1, wherein the atraumatic anchor comprises a helical coil having an atraumatic tip.

9. The LAAC device of claim 8, wherein the helical coil comprises one or more retention bumps.

10. The LAAC device of claim 8, wherein the helical coil has a distally increasing diameter.

11. The LAAC device of claim 1, wherein the atraumatic anchor comprises two or more intertwined helical coils.

12. The LAAC device of claim 11, wherein the two or more intertwined helical coils each have a constant diameter.

13. The LAAC device of claim 11, wherein the two or more intertwined helical coils each have a distally increasing diameter.

14. The LAAC device of claim 1, wherein the atraumatic anchor comprises a plurality of curved members extending radially outwardly from a central point.

15. The LAAC device of claim 14, wherein the plurality of curved members define a proximally increasing diameter.

16. The LAAC device of claim 1, further comprising an expandable coating on at least part of the atraumatic anchor.

17. The LAAC device of claim 1, wherein the expandable occlusive element is keyed to a delivery device component.

18. A left atrial appendage closure (LAAC) device, comprising:
an expandable foam element movable from a collapsed configuration for delivery to an expanded configuration for deployment; and
an atraumatic anchor engaged with the expandable foam element, the atraumatic anchor adapted to engage pectinate within the left atrial appendage (LAA) in order to secure the expandable occlusive element within the LAA.

19. The LAAC device of claim 17, wherein the atraumatic anchor comprises one or more helical coils.

20. A left atrial appendage closure (LAAC) device, comprising:
an expandable foam element movable from a collapsed configuration for delivery to an expanded configuration for deployment; and
an atraumatic anchor engaged with the expandable foam element, the atraumatic anchor comprising:
a base portion adapted to be secured to the expandable foam element;
a coil adapted to extend distally from the base portion and engage with pectinate within the left atrial appendage (LAA); and
an atraumatic tip secured to a distal end of the coil.
